(19) United States
(12) Plant Patent Application Publication
(10) Pub. No.: US 2012/0073024 P1
(43) Pub. Date: Mar. 22, 2012

(54) Alyogyne plant named 'Delightfully Double'

(50) ;

(76) Inventor: Steven Eggleton, (US)

(21) Appl. No.: 13/317,859

(22) Filed: Oct. 31, 2011

(30) Foreign Application Priority Data
Sep. 21, 2010 (AU) 2010/218

(51) Int. Cl. A01H 5/00

(52) U.S. Cl. PLT/263.1

(57) ABSTRACT

A new and distinct Alyogyne cultivar named ‘DELIGHTFULLY DOUBLE’ is disclosed, characterized by red-purple double flowers, moderately rapid growth and drought resistance in established plants. The new variety is a Alyogyne, typically produced as an outdoor ornamental plant.

### LATIN NAME OF THE GENUS AND SPECIES

Alyogyne huegeli×hakefolia

### VARIETY DENOMINATION

‘DELIGHTFULLY DOUBLE’

### BACKGROUND OF THE INVENTION

The applicant claims priority to Australian Plant Breeder's Rights Application 2010/218, lodged Sep. 21, 2010. The new cultivar is a product of a planned breeding program. The objective of the breeding program was to develop a new Alyogyne variety with double flowers. The new variety originated from a cross pollination of unpatented, unnamed variety of Alyogyne huegelii as the seed parent and unpatented, unnamed variety Alyogyne hakefolia as the pollen parent. The crossing was made in November 2002 at a commercial nursery in Wonga Park, Victoria, Australia.

The new variety was discovered by the inventor, Steve Eggleton, a citizen of Australia, in March 2004 in a group of seedlings resulting from the crossing. The new cultivar was found in the same commercial greenhouse in Wonga Park, Australia. After identifying the new variety as a potentially interesting selection, the inventor continued confidential trial growing of ‘DELIGHTFULLY DOUBLE’ for 4 years, assessing stability of the double flower type.

Asexual reproduction of the new cultivar ‘DELIGHTFULLY DOUBLE’ was first performed at a commercial nursery Wonga Park, Victoria, Australia by cuttings in March of 2004. Subsequent propagation by tissue culture has shown that the unique features of this cultivar are stable and reproduced true to type.

### SUMMARY OF THE INVENTION

The cultivar ‘DELIGHTFULLY DOUBLE’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘DELIGHTFULLY DOUBLE.’ These characteristics in combination distinguish ‘DELIGHTFULLY DOUBLE’ as a new and distinct Alyogyne cultivar:

1. Unique double flowers.

2. Red-Purple flower color.

3. Rapid growth habit.

4. Drought resistance in established plants.

### PARENT COMPARISON

Plants of the new cultivar ‘DELIGHTFULLY DOUBLE’ are similar to the seed parent, Alyogyne huegelii in most horticultural characteristics. The new variety, however, produces double flowers, compared to the single flowers produced by the seed parent. Additionally, ‘DELIGHTFULLY DOUBLE’ produces flowers with a funnelform shape, compared to the flattened shape of Alyogyne huegelii.

Plants of the new cultivar ‘DELIGHTFULLY DOUBLE’ are similar to the pollen parent Alyogyne hakefolia in most horticultural characteristics The new variety, however, produces double flowers, compared to the single flowers produced by the pollen parent. Additionally, ‘DELIGHTFULLY DOUBLE’ produces foliage with broad leaf lobes, compared to the narrow leaf lobes typical of Alyogyne huegelii.

### COMMERCIAL COMPARISON

‘DELIGHTFULLY DOUBLE’ can be compared to compared to the commercial variety Alyogyne huegelii ‘West Coast Gem’ unpatented. Plants of ‘West Coast Gem’ are similar to plants of ‘DELIGHTFULLY DOUBLE’ in most horticultural characteristics, however, plants of ‘DELIGHTFULLY DOUBLE’ produce double flowers that are red-purple in color, whereas ‘West Coast Gem’ produces single flowers that are purple. Additionally, flower shape of ‘West Coast Gem’ is flattened, whereas ‘DELIGHTFULLY DOUBLE’ produces funnelform shaped flowers.

‘DELIGHTFULLY DOUBLE’ can be compared to compared to the commercial variety Alyogyne hadkeifolia ‘Melissa Anne’ unpatented. Plants of ‘Melissa Anne’ are similar to plants of ‘DELIGHTFULLY DOUBLE’ in most horticultural characteristics, however, plants of ‘DELIGHTFULLY DOUBLE’ produce double flowers, and foliage with deep lobes. Plants of ‘Melissa Anne’ produce single flowers with shallow leaf lobes.

### BRIEF DESCRIPTION OF THE PHOTOGRAPHS

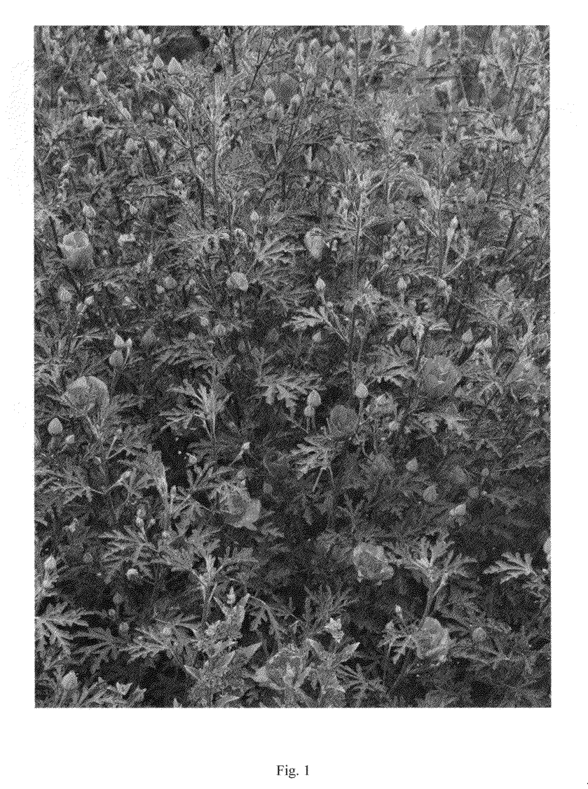

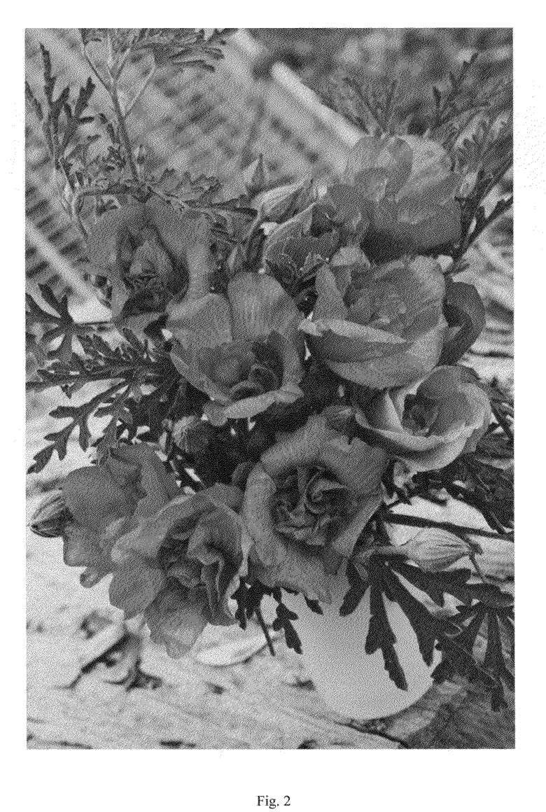

The accompanying photograph in FIG. 1 illustrates in full color a typical plant of ‘DELIGHTFULLY DOUBLE’ grown outdoors in Wonga Park, Australia. This plant is approximately 18 months old, shown planted in the ground. FIG. 2 illustrates in full color a close up of the flowers of ‘DELIGHTFULLY DOUBLE. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2001, except where general terms of ordinary dictionary significance are used. The following observations and measurements describe ‘DELIGHTFULLY DOUBLE’ plants grown outdoors in Camarillo, Calif., USA. Temperatures ranged from 10° C. to 25° C. at night to 12° C. to 37° C. during the day. No artificial light, photoperiodic treatments were given to the plants. Measurements and numerical values represent averages of typical plant types.
Botanical classification: Alyogyne huegeli×hakefolia ‘DELIGHTFULLY DOUBLE’
Propagation:
   Time to initiate roots.—Approximately 45 days at approximately 25° C. soil temperature.
   Time to produce a fully rooted plantlet.—Approximately 12 weeks at approximately 20 to 30° C. soil temperature.
   Root description.—Thick, fleshy roots with fine, fibrous lateral roots. Color near Orange-White 158D.
Plant:
   Growth habit.—Upright tender perennial.
   Height.—Approximately 50 cm.
   Plant spread.—Approximately 35.
   Pot size of plant described.—1 gallon.
   Growth rate.—Moderate.
   Branching characteristics.—Moderate branching after a pinch.
   Length of primary lateral branches.—15 cm.
   Diameter of lateral branches.—0.3 cm.
   Quantity of primary lateral branches.—Approximately 60.
   Characteristics of primary lateral branches.—Form: Round Color: Near RHS Yellow-Green 147A Texture: Sparsely pubescent, with lenticels. Lenticels: Approximately 0.2 cm long and 0.1 cm wide. Colored near Green-White 157A. Strength: Moderately strong, somewhat flexible.
   Internode length.—Average 4.7 cm.
   Age of plant described.—Approximately 6 months.
Foliage:
   Leaf.—Arrangement: Opposite Average Length(excluding petiole): Approximately 3.5 cm. Average Width: Approximately 4.1 cm. Shape of blade: Deeply lobed deltoid Aspect: Flat. Apex: Acute Base: Truncate Margin: Entire. Appearance: Matte, leathery Texture of top surface: Non-pubescent, rough. Texture of bottom surface: Non-pubescent, rough. Color: Young foliage upper side: Near Green 137A Young foliage under side: Near Green 137B Mature foliage upper side: Near Green137A Mature foliage under side: Near Green 137A.
   Venation.—Type: Pinnate. Venation coloration upper side: Near Green 137C Venation coloration under side: Near Green 137C.
   Petiole.—Length: Average 2.5 cm Width: Approximately 0.2 cm Color: Near RHS Green 137A Strength: Very strong Other: Somewhat pubescent.
Inflorescence:
Flower:
   Natural flowering season.—Spring through Summer.
   Days to flowering from rooted cutting.—Approximately 120.
   Inflorescence and flower type and habit.—Single occurring, double funnelform flowers emerging from leaf axils.
   Rate of flower opening.—3 to 5 days from bud to fully opened flower.
   Flower longevity on plant.—About 5 to 7 days.
   Persistent or self-cleaning.—self-cleaning.
   Bud.—Shape: Elongated sphere Length: Approximately 3.4 cm Diameter: 2.1 cm Color: Near RHS Red-Purple 72B.
   Flower size.—Diameter: 4.8 cm. Length: 5.0 cm.
   Corolla/petals.—Arrangement: Rotate Petal Quantity: 5 large outer petals, 7 to 9 small inner petals Large petals: Length: 4.5 cm. Width: 2.4 cm. Small petals: Length: 2.5 cm. Width: 1.9 cm. Texture: Glabrous Appearance: Somewhat shiny Apex: Obtuse shape: Orbicular Margin: Entire. Aspect: Somewhat ruffled along margin.
   Color.—When opening: Upper surface: Near RHS Red-Purple 72A Lower surface: Near RHS Red-Purple 72A, 72C closer to base Fully opened: Upper surface: Near RHS Red-Purple 72B Lower surface: Near RHS Red-Purple 72A, 72S closer to base Ageing: Upper surface: Near RHS Purple 77A Lower surface: Near RHS Purple 77A, base near 77D.
   Calyx/sepals.—Quantity per flower: 5 Shape: Narrow deltoid Length: 2.0 cm Width: 0.7 cm Apex: Acute Base: Fused Margin: Entire Texture: Pubescent Color: Upper Surface: Near RHS Green 137A Lower Surface: Near RHS Green 137A.
   Peduncle.—Length: Average 3.5 cm. Diameter: Approximately 0.2 cm. Color: Near RHS Green 137A Orientation: Approximately 45 degree angle from leaf axil Pubescence: Pubescent.
   Fragrance.—Sweet.
Reproductive organs:
   Stamens.—Number: 4 Filament length: Approximately 0.3 cm.
   Anthers.—Shape: Globular Length: Approximately 0.1 cm. Color: Near RHS White N155C Pollen: Pollen production has not been observed after extensive trials.
   Pistil.—Number: 1 Length: Approximately 0.9 cm. Style: Length: 0.1 cm. Color: Near RHS White N155C Stigma: Shape: linear Color: Near RHS White N155C Ovary: Ovary Color: Near RHS White 155A Ovary length: Approximately 0.8 cm.
Other characteristics:
   Disease resistance.—Neither resistance nor susceptibility to the normal diseases and pests of Alyogyne has been observed.
   Drought tolerance and cold tolerance.—The new cultivar is a typical Alyogyne, cold tolerant to approximately −7° C. and tolerates drought very well.
   Fruit/seed production.—Not observed after extensive trials.

What is claimed is:

1. A new and distinct cultivar of Alyogyne plant named ‘DELIGHTFULLY DOUBLE’ as herein illustrated and described.
